(19) United States
(12) Patent Application Publication
(10) Pub. No.: US 2022/0370293 A1
(43) Pub. Date: Nov. 24, 2022

(54) COMBINED BIOLOGICAL SAMPLING AND INJECTION ASSEMBLIES AND ASSOCIATED DEVICES, SYSTEMS, AND METHODS

(71) Applicant: Twenty Twenty Therapeutics LLC, South San Francisco, CA (US)

(72) Inventors: Alexandre R. Tumlinson, South San Francisco, CA (US); Ellen Kaplan, South San Francisco, CA (US)

(21) Appl. No.: 17/750,563

(22) Filed: May 23, 2022

Related U.S. Application Data
(60) Provisional application No. 63/192,480, filed on May 24, 2021.

(51) Int. Cl. A61J 1/20; A61J 1/14

(52) U.S. Cl. CPC A61J 1/2096; A61J 1/201; A61J 1/2055; A61J 1/1406

(57) ABSTRACT

A sampling device includes a container having a body defining a chamber and an opening to the chamber. A septum is bonded to the container to cover the opening and hermetically seal the chamber. The septum has an adherent layer with a first thickness and including a material configured to adhere to a rim of the container to provide a hermetic seal with the container. A metallic foil layer having a second thickness is coupled to the adherent layer. An elastomeric layer having a third thickness is coupled to the metallic foil layer to position the metallic foil layer between the adherent layer and the elastomeric layer. The third thickness is greater than the sum of the first thickness and the second thickness.

### RELATED APPLICATIONS

This application claims the benefit of U.S. Provisional Appln. Ser. No. 63/192,480, filed May 24, 2021, the entirety of which is incorporated by reference herein.

### TECHNICAL FIELD

The subject matter described herein relates to devices, systems, and methods for injection of substances into, and sampling of, aqueous and vitreous humors of the eye. The disclosed intravitreal injection and sampling device has particular but not exclusive utility for diagnosis and treatment of ophthalmic disorders in humans.

### BACKGROUND

Vitreous humor is a colorless, gelatinous fluid within an eye or eyeball of humans or other vertebrates composed of approximately 98-99% water with trace amounts of hyaluronic acid, glucose, anions, cations, ions, and a fine network of collagen. Vitreous humor provides support to the surrounding structures of the eye, absorbs mechanical trauma, and provides circulation and regulation of oxygen, metabolites and nutrients. It is produced largely by cells of the ciliary body. Changes in vitreous structure that occur with aging, are important in the pathogenesis of many vitreoretinal diseases.

Intraocular pressure (TOP) quantifies the pressure of the vitreous humor inside the eye. Many individuals suffer from disorders, such as glaucoma, that are associated with chronic heightened IOP. Over time, heightened IOP can cause damage to the optical nerve of the eye, leading to loss of vision.

Presently, treatment of ophthalmic disorders mainly involves periodically administering pharmaceutical agents to the eye. These drugs can be delivered by, for example, intravitreal injection. Intravitreal injection is one of the most common surgical procedures performed in ophthalmology today. A variety of drugs are delivered directly to the clear vitreous gel that supports the globe of the eye. These drugs act directly in the vitreous or in the surrounding retinal tissues over the following months. For example, intravitreal injection is a common route of delivery for vascular endothelial growth factor inhibiting (anti-VEGF) proteins, which are highly potent compounds tolerated at high doses, with intravitreal half-lives about one week. Anti-VEGF biologics and steroids are the most commonly administered drugs by this route. These drugs may be administered on a chronic basis.

One recommended procedure for intravitreal injection includes preparation of an injection needle, topical anesthesia and disinfection of the eye surface, holding the eye open with a lid speculum or other means, optional lateral dislocation of the conjunctiva at the injection site, and insertion of the needle a few mm lateral to the limbus to approximately the full depth of the needle, injecting the drug, withdrawing the needle, and allowing the conjunctiva to cover the injection site. Post injection care typically includes a basic verification of functional vision such as requesting the patient to count the number of fingers shown by the doctor. This functional test verifies that acute TOP increase due to injection has not impacted the optic nerve head in a way that requires immediate relief.

Another important ophthalmic procedure is vitreous sampling. Vitreous sampling may inform various aspects of eye care. Samples of vitreous may be analyzed for cellular content and extracellular structure by histology or immunologic analysis. Histology can, for example, provide a definitive diagnosis for the type of infection causing endophthalmitis.

Identification of the type of immune cells present and the immune mediator proteins expressed may inform the treatment of uveitis. Identification of the amount of VEGF present in the vitreous may give an indication of how likely imminent neovascularization is to occur or how likely it is that VEGF compounds are responsible for an observed case of neovascularization. Non-responders to anti-VEGF treatment remains one of the most troublesome aspects of treating neovascularization in exudative, age-related vascular degeneration (also known as wet AMD) and diabetic retinopathy.

Two common methods of vitreous sampling—with a cutter or with needle aspiration—appear to be approximately equivalent for the purposes of protein analysis. A state of the art miniature cutting tool may be delivered through a 23-gauge trocar. Needle aspiration may be performed with needles as small as 30-gauge (about half the diameter of 23 gauge). Fine gauge may increase the probability of a dry tap and/or change the properties of the aspirated material by acting as a filter. Small gauge may have an advantage in that traction may not be introduced on the gel matrix because the gel matrix cannot be pulled into the small needle bore. Vitreous samples are typically frozen or otherwise stabilized so that they can be processed in a laboratory outside of the operating room or ophthalmic office setting.

Injection of therapeutic doses of medication into the vitreous or aqueous humor inside the eye can increase TOP by as much as 25 mmHg, which is substantially greater than threshold levels that are considered potentially harmful. Evidence shows that while such TOP increases are transient, they are in fact associated with an iatrogenic glaucoma resulting in measurable loss of nerve fiber layer and visual function over a course of only several treatments in patients with ‘normal’ resting TOP. See Saxena, S., Lai, T. Y., Koizumi, H. et al., “Anterior chamber paracentesis during intravitreal injections in observational trials: effectiveness and safety and effects,” International Journal of Retina and Vitreous, 5, 8 (2019). Therefore, it is sometimes desirable to remove a small volume of humor (whether aqueous, vitreous or both) from the eye before injecting a comparable volume of medication. However, removal of a volume of humor may result in insufficient pressure, which can also be harmful to the eye.

Therefore, in the case of diagnostic sampling of humors, it may be necessary or beneficial to inject a volume of fluid (whether medicated or otherwise) to replace the withdrawn humors. In either case, care must be taken to ensure that the removed and injected volumes are comparable, and in either case, two separate procedures (a sampling procedure and an injection procedure) are typically required.

The information included in this Background section of the specification, including any references cited herein and any description or discussion thereof, is included for technical reference purposes only and is not to be regarded as subject matter by which the scope of the disclosure is to be bound.

### SUMMARY

Disclosed herein are devices, assemblies, and subassemblies for performing biological sampling and injection procedures. An assembly may include a syringe containing or configured to contain, a liquid drug or pharmaceutical agent, an adapter, a working barrel, and a sample container positioned within the working barrel. The sample container or chamber may include a cylindrical body and at least one multi-layer septum providing a hermetic seal for at least one opening of the cylindrical body. The multi-layer construction of the septum may include a metal foil layer and an elastomeric layer to reduce gas permeability and to maintain a vacuum inside the sample container. The assembly may provide for both biological sampling and drug injection with a stepwise motion of the drug syringe so that a biological sample is obtained when the syringe is moved to a first position within the barrel, and the drug is injected when the syringe is moved to a second position within the barrel. In some aspects, the combined biological sampling and injection assembly may be controlled with one hand by a physician, increasing the efficiency and decreasing the complexity of the procedure.

An example sampling device includes a container having a body, and an opening, where the body defines a chamber. The sampling device further includes a septum bonded to the container at the opening to hermetically seal the chamber. The septum includes an adherent layer formed from a material configured to adhere to a rim of the container to provide a hermetic seal with the container. A metallic foil layer is coupled to the adherent layer. An elastomeric layer is coupled to the metallic foil layer to position the metallic foil layer between the adherent layer and the elastomeric layer.

In another aspect, taken alone or in combination with any other aspect, the adherent layer may include a first thickness, the metallic foil layer may include a second thickness, and the elastomeric layer may include a third thickness. The third thickness is greater than the sum of the first thickness and the second thickness.

In another aspect, taken alone or in combination with any other aspect, the opening of the container is a distal opening. The container further includes a proximal opening. In some embodiments, the sampling device further includes a proximal septum bonded to the container at the proximal opening. The proximal septum includes an adherent layer formed from a material configured to adhere to a proximal rim of the container to provide a hermetic seal with the container. A metallic foil layer is coupled to the adherent layer of the proximal septum. An elastomeric layer is coupled to the metallic foil layer of the proximal septum to position the metallic foil layer of the proximal septum between the adherent layer and the elastomeric layer of the proximal septum. The adherent layer may include a heat-activated material configured to bond with the rim of the container in response to applying heat. The adherent layer may include an adhesive.

In another example, a system is provided for use with a syringe for obtaining a biological sample and delivering a pharmaceutical agent to a patient. The system includes a barrel having a hollow body defining an interior region. An adapter is positioned within the interior region and has a proximal connecter configured to couple the adapter to a distal end of the syringe. A first septum is disposed at a distal end of the adapter. An evacuated container is slidably received within the interior region distally from the adapter and includes a body defining a chamber configured to contain the biological sample and an opening extending to the chamber. A second septum covers the opening. A two-sided needle has a proximal end disposed within the barrel distally from the evacuated container and a distal end positioned for insertion into the patient. The evacuated chamber is movable within the barrel to a first position placing the proximal end of the needle within the chamber to draw the biological sample from the patient into the chamber and to a second position in which the proximal end of the needle passes entirely through the evacuated chamber and through the first septum to deliver the pharmaceutical agent from the syringe to the patient.

In another aspect, taken alone or in combination with any other aspect, the evacuated container is movable in the same direction relative to the barrel to reach both the first and second positions.

In another aspect, taken alone or in combination with any other aspect, the opening of the container is a distal opening and the second septum includes an adherent layer having a material configured to adhere to a distal rim of the container to provide a hermetic seal with the container. A metallic foil layer is coupled to the adherent layer of the second septum. An elastomeric layer is coupled to the metallic foil layer of the second septum to position the metallic foil layer of the second septum between the adherent layer and the elastomeric layer of the second septum.

In another aspect, taken alone or in combination with any other aspect, the adherent layer has a first thickness, the metallic foil layer has a second thickness, and the elastomeric layer has a third thickness. The third thickness is greater than the sum of the first thickness and the second thickness.

In another aspect, taken alone or in combination with any other aspect, the evacuated container further includes a third septum bonded to the container at a proximal opening opposite and aligned with the distal opening to cover the proximal opening. The third septum includes an adherent layer having a material configured to adhere to a proximal rim of the container to provide a hermetic seal with the container. A metallic foil layer is coupled to the adherent layer of the third septum. An elastomeric layer is coupled to the metallic foil layer of the third septum. The metallic foil layer of the third septum is disposed between the adherent layer and the elastomeric layer of the third septum.

In another aspect, taken alone or in combination with any other aspect, the adherent layer of the third septum has a fourth thickness, the metallic foil layer of the third septum has a fifth thickness, and the elastomeric layer of the third septum has a sixth thickness. The sixth thickness is greater than the sum of the fourth thickness and the fifth thickness.

In another aspect, taken alone or in combination with any other aspect, the adapter has an o-ring around a perimeter of the adapter. An outer diameter of the o-ring corresponds to an inner diameter of the barrel.

In another aspect, taken alone or in combination with any other aspect, the barrel further includes a tactile feature within the interior region and cooperating with the o-ring to indicate that the evacuated container is at the first position.

In another aspect, taken alone or in combination with any other aspect, the proximal end of the two-sided needle has a beveled opening.

In another aspect, taken alone or in combination with any other aspect, the barrel defines a longitudinal axis and at least the proximal end of the two-sided needle is offset from the longitudinal axis. The beveled opening is configured to cause, in response to puncturing the second septum of the evacuated container, a deflection of the proximal portion of the two-sided needle toward the longitudinal axis.

In yet another example, an adapter configured to couple to a syringe is provided. The adapter includes a receptacle configured to receive a portion of the syringe. A proximal connector on the receptacle is configured to mate with a corresponding connector feature of the syringe. The proximal connector is a first type of connector. The proximal connector includes a channel extending along a needle axis. A septum disposed at a distal end of the receptacle is configured to mate with a needle assembly that includes a second type of connector different from the first type of connector

In another aspect, taken alone or in combination with any other aspect, the receptacle has a cylindrical body with at least one lateral window extending therethrough.

In another aspect, taken alone or in combination with any other aspect, the proximal connector includes a female luer-lock connector. The septum includes a multi-puncture septum.

In another aspect, taken alone or in combination with any other aspect, the receptacle has a first width and the adapter further may include a distal neck coupled to the septum and having a second width smaller than the first width.

In another aspect, taken alone or in combination with any other aspect, a relief needle assembly is provided which includes a needle having a proximal portion and a distal portion. A cap is positioned around the distal portion. The spacer is coupled to the cap and extend proximally from the cap. The spacer is disposed at least partially around the proximal portion of the needle and configured to releasably couple to the distal connector.

In another aspect, taken alone or in combination with any other aspect, the spacer is configured to releasably couple to the distal connector such that the proximal portion of the needle punctures the septum.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a perspective view of a combined biological sampling and injection assembly performing an ophthalmic sampling and injection procedure, according to aspects of the present disclosure.

FIG. 2 is an elevation view of a combined biological sampling and injection assembly, according to some aspects of the present disclosure.

FIG. 3A is an elevation view of a relief needle and adapter subassembly of the combined biological sampling and injection assembly, according to some aspects of the present disclosure.

FIG. 3B is a perspective exploded view of the relief needle and adapter subassembly shown in FIG. 3A, according to some aspects of the present disclosure.

FIG. 3C is an elevation view of an adapter of the combined biological sampling and injection assembly shown in FIG. 3A, according to some aspects of the present disclosure.

FIG. 3D is a cross-sectional view of the adapter of FIG. 3C coupled to a syringe of the biological sampling and injection assembly shown in FIG. 3A, according to some aspects of the present disclosure.

FIG. 4A is an elevation view of a working barrel of a biological sampling and injection assembly, according to some aspects of the present disclosure.

FIG. 4B is a perspective exploded view of the working barrel of a biological sampling and injection assembly shown in FIG. 4A, according to some aspects of the present disclosure.

FIG. 5 is a cross-sectional view of an evacuated sample container with multi-layer pierceable septa, according to some aspects of the present disclosure.

FIG. 6 is a flow diagram of a method for performing a biological sampling and injection procedure using a combined biological sampling and injection assembly, according to some aspects of the present disclosure.

FIG. 7A is a disassembled view of a relief needle and adapter subassembly of the combined biological sampling and injection assembly, according to some aspects of the present disclosure.

FIG. 7B is an elevation view of the relief needle and adapter subassembly in a first step of a method for performing the biological sampling and injection procedure, according to aspects of the present disclosure.

FIG. 7C is an elevation view of a drug syringe and adapter subassembly in a second step of the method for performing the biological sampling and injection procedure, according to aspects of the present disclosure.

FIG. 7D is an elevation view of a drug syringe and adapter subassembly, and a working barrel and sampling subassembly in a third step of the method for performing a biological sampling and injection procedure, according to aspects of the present disclosure.

FIG. 7E is an elevation view of a combined biological sampling and injection assembly in a fourth step of the method for performing the biological sampling and injection procedure, according to aspects of the present disclosure.

FIG. 7F is an elevation view of the combined biological sampling and injection assembly in a fifth step of the method for performing the biological sampling and injection procedure, according to aspects of the present disclosure.

FIG. 8 is an elevation view of a multi-puncture pre-filled drug syringe of a combined biological sampling and injection assembly, according to aspects of the present disclosure.

FIG. 9A is a diagrammatic view of a working barrel including an angularly deflected needle, according to aspects of the present disclosure.

FIG. 9B is a diagrammatic view of a working barrel including a laterally deflected needle, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Disclosed herein are devices, assemblies, and subassemblies for performing biological sampling and injection procedures. An assembly may include a syringe containing, on configured to contain, a liquid drug or pharmaceutical agent, an adapter, a working barrel, and a sample container positioned within the working barrel. The assembly may provide for both biological sampling and drug injection with a stepwise motion of the drug syringe, so that a biological sample is obtained when the syringe is moved distally to a first position within the barrel, and the drug is injected when the syringe is moved distally to a second position within the barrel. In some aspects, the combined biological sampling and injection assembly may be controlled with one hand by a physician, increasing the efficiency and decreasing the complexity of the procedure.

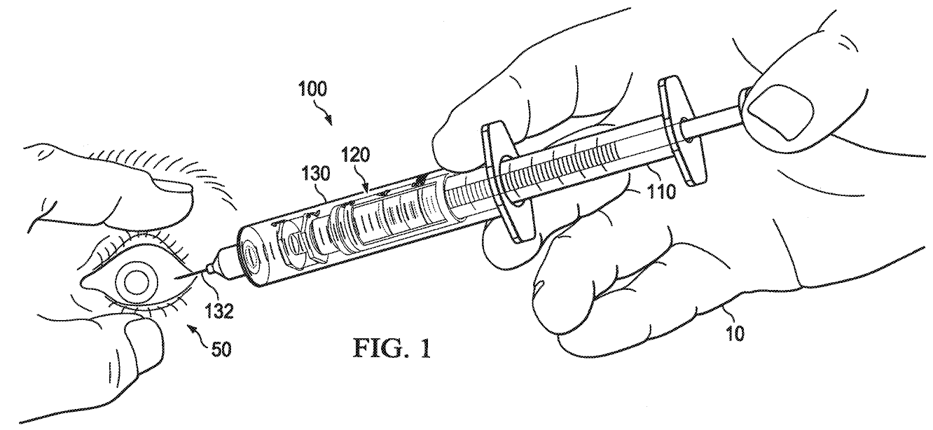

FIG. 1 is an example combined biological sampling and injection assembly 100 being used by a physician's hand 10 to extract a sample from a patient's eye 50, and deliver a drug or pharmaceutical agent to the eye 50. In an exemplary embodiment, the assembly 100 may be used to obtain a sample of the vitreous humor and to inject a drug into the vitreous humor.

However, the assembly 100 may be used in other scenarios instead of or in addition to the ophthalmic procedure shown in FIG. 1. This can include, for example, obtaining blood samples and injecting the drug into the patient's blood vessels. In some aspects, the assembly 100 may be configured to inject a volume of drug that is equal to or approximately equal to, a volume of the sample obtained from the patient's anatomy.

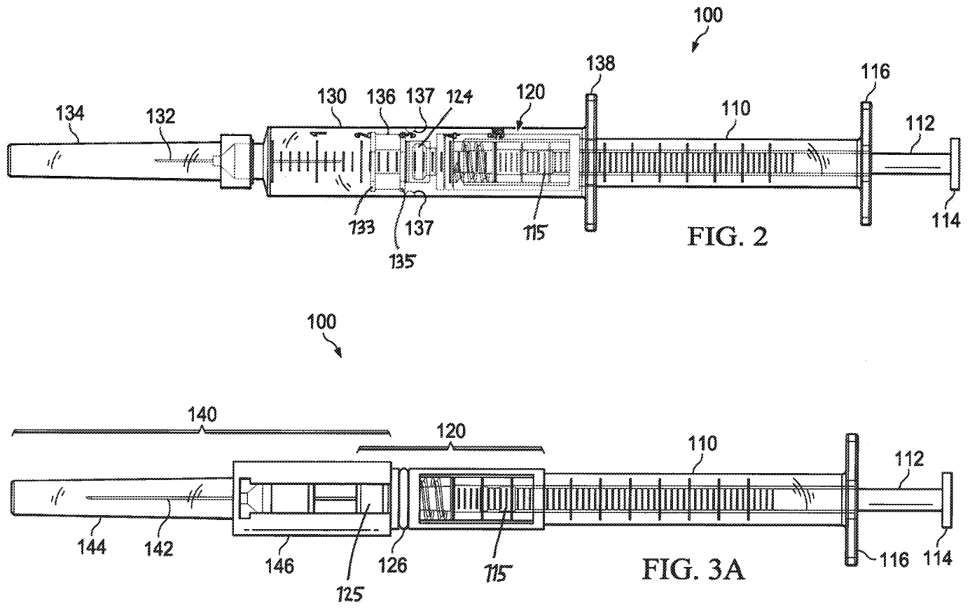

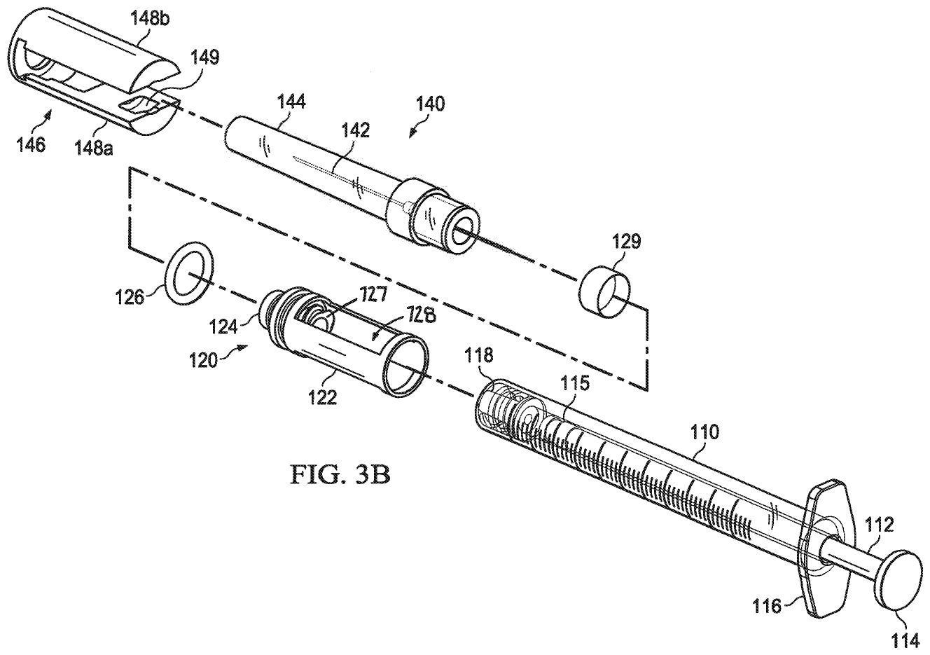

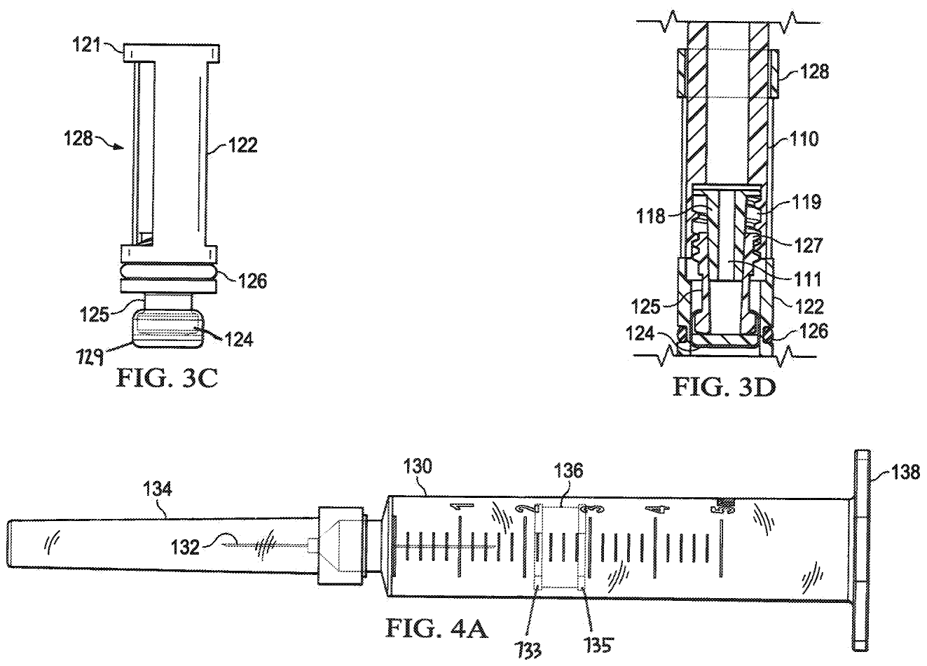

Referring to FIGS. 2-3, the assembly 100 includes a drug syringe 110 and an adapter 120 coupled to a distal end of the drug syringe 110. A working barrel 130 slidably receives the drug syringe 110, adapter 120, and an evacuated sampling chamber or device 136. To this end, the syringe 110 and/or the adapter 120 may include one or more features that form an interference fit with an interior surface of the working barrel 130. The friction resulting from the interference fit may allow the syringe 110 and the adapter 120 to be moved longitudinally within the working barrel 130 while providing some resistance and stability so that the syringe 110 and/or the adapter 120 do not freely move within the working barrel 130 without a force applied by the physician's hand 10. In other embodiments, the friction associated with the interference fit may be sufficiently low such that the syringe 110 and/or the adapter can freely move within the working barrel 130 even without a force applied by the physician's hand 10. A needle 132 is coupled to a distal end of the working barrel 130.

The syringe 110 can be prefilled with a volume of a liquid pharmaceutical agent, which is indicated generally by the graduated marks on the outside of the syringe body. The syringe 110 may be formed of a transparent material, such as a transparent polymer or plastic. For example, the syringe 110 may be formed of transparent polycarbonate, polyester, polypropylene, nylon, cyclic olefin copolymer and/or any other suitable polymer or glass.

The syringe 110 includes a plunger 112 having a push pad 114 at a proximal end of the plunger 112. A stopper 115 is coupled to a distal end of the plunger 112, and forms a slidable seal with the interior surface of the syringe body. A first finger grip 116 is provided at a proximal end of the syringe 110. A connector 118 is provided at a distal end of the syringe 110 and defines a fluid channel 111 (see FIG. 3D).

The adapter 120 includes a connector 125 at its distal end configured to provide a different interface for accessing the drug within the syringe 110. With this in mind, the adapter 120 may be provided with a multi-puncture interface or septum 124 such that the drug within the syringe 110 can be accessed multiple times.

One example adaptor 120 is illustrated in FIGS. 3B-3D. The adapter 120 includes a generally cylindrical receptacle 122 configured to receive a distal end of the syringe 110 and a proximal connector 127 configured to mate with the corresponding distal connector 118 of the syringe 110. To this end, the connector 127 of the adapter 120 may include a luer-lock connector, such as a female luer-lock connector or a male luer-lock connector. Similarly, the connector 118 of the syringe 110 may include a luer-lock connector, such as a male luer-lock connector or a female luer-lock connector. However, other connectors are also contemplated, including threaded or non-threaded connectors.

The distal connector 125 includes a head portion and a narrow neck. The multi-puncture septum 124 is coupled to the head portion of the connector 125. In the illustrated embodiment, the multi-puncture septum 124 is crimped onto the head portion and the neck portion. In particular, a crimp cap 129 can be used to crimp the multi-puncture septum 124 around the head and neck portions of the adaptor 120.

The receptacle 122 includes one or more lateral, radially extending window portions 128 and a proximal ring portion 121. In some aspects, the receptacle 122 of the adapter 120 may be formed of a non-transparent material. Accordingly, the window portions 128 may provide visibility of the syringe so that the physician can monitor the volume of fluid remaining in the syringe 110. The proximal ring portion 121 may provide structural integrity and rigidity to the receptacle 122. The adapter 120 further includes an o-ring 126. The outer diameter of the o-ring 126 may be selected or otherwise configured based on an interior diameter of the working barrel 130. In that regard, the o-ring 126 may provide for a sliding interference fit with the interior surface of the working barrel 130.

Referring to FIG. 2, the working barrel 130 includes a transparent body which provides visibility to the components (e.g., syringe 110, adapter 120, sampling chamber 136) therein. For example, the working barrel 130 may be formed of transparent polycarbonate, polyester, polypropylene, nylon, polyvinyl chloride, cyclic olefin copolymer and/or any other suitable polymer.

The working barrel 130 further includes graduated marks to allow the physician to monitor the position and movement of the components within the working barrel 130. The working barrel 130 includes a second finger grip 138, which can be gripped by a physician (e.g. as shown in FIG. 1). For example, the physician may use the second finger grip 138 to hold the working barrel 130 stationary while the syringe 110, adapter 120, and sampling chamber 136 are pushed distally into the working barrel 130 by pushing on the push pad 114 of the syringe 110. In some aspects, the finger grips 116, 138 may be referred to as flanges. The finger grips 116, 138 may be integrally formed (e.g. injection molded) with the syringe body and the working barrel 130, respectively.

The working barrel 130 also includes a tactile feature 137 on the interior surface of the working barrel 130, which is configured to engage and/or interface with one or more surfaces of the adapter 120, the syringe 110, and/or the sampling chamber 136. In the illustrated embodiment, the tactile features 137 includes an annular ridge or projection and extending inward into the working barrel 130.

A needle 132 is coupled to a distal end of the working barrel 130. A cap 134 is coupled to a distal end of the working barrel 130 to provide protection for the needle 132 when the working barrel 130 is not in use. The needle 132 may include a two-sided needle configured to be coupled to a distal opening or nozzle of the working barrel 130 such that a proximal portion of the needle is disposed within the working barrel 130.

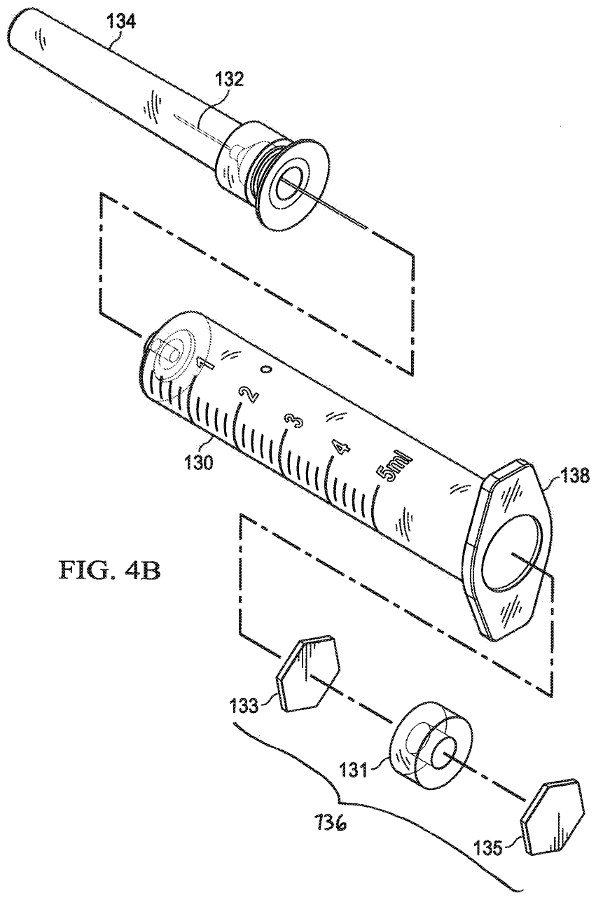

Referring to FIG. 4B, the sampling chamber 136 may include a cylindrical or tubular body or container 131 having openings at the proximal and distal ends. One or both of the proximal and distal opening(s) of the sampling chamber 136 are covered by corresponding a respective septa 133, 135. The septa 133, 135 may be configured to maintain a negative pressure or vacuum inside the sampling chamber 136 relative to the exterior environment. In some aspects, the sampling chamber 136 is configured to maintain a pressure lower than the pressure of the vitreous humor within the patient's eye 50, for example.

The cylindrical or tubular body of the sampling chamber 136 may be transparent to allow the physician to observe the material filling the chamber 136. The sampling chamber 136 is sized, shaped, and otherwise structurally configured to fit within the working barrel 130, and may be configured to form an interference fit with the interior surface of the working barrel 130. For example, the sampling chamber 136 may be configured to remain stationary within the working barrel 130 until a force is applied to the sampling chamber 136 (e.g., via the syringe 110) relative to the working barrel 130.

To this end, the septa 133, 135 can have a width or outer diameter that is equal to or greater than an interior width or diameter of the working barrel 130. Alternatively, the sampling chamber 136 floats freely within the working barrel 130, such that the sampling chamber 136 can freely slide longitudinally relative to the working barrel 130. Although shown as hexagonal, it will be understood that the proximal and distal septa 135, 133 may be circular, rectangular, octagonal, elliptical or any other suitable shape.

In any case, the distal end of the adapter 120 is configured to push the sampling chamber 136 distally within the working barrel 130. In this regard, the sampling chamber 136 may not be attached or connected to the adapter 120, but rather is configured to be moved within the working barrel 130 by advancing the syringe 110 and adapter 120 distally to push the sampling chamber 136 into the working barrel 130 toward the needle 132. In other embodiments, the sampling chamber 136 may be connected to, adhered to, welded tube or otherwise coupled to the adapter 120 and/or the syringe 110. Advancing the syringe 110, adapter 120, and sampling chamber 136 distally toward the needle 132 causes the proximal end of the needle 132 to penetrate, in succession, the sampling chamber 136 and the drug reservoir of the syringe 110.

FIGS. 3A-3B further illustrate a relief needle and adapter subassembly 140 that can be used in combination with the biological sampling and injection assembly 100. The relief needle subassembly 140 includes a needle 142, a cap 144, and a spacer 146. The relief needle subassembly 140 is configured such that the proximal end or opening of the needle 142 punctures the multi-puncture septum 124 on the adapter 120. Since the multi-puncture septum 124 can be crimped onto the distal connector 125, the proximal end of the needle 142 can be placed in fluid communication with the fluid or drug inside the syringe 110. The spacer 146 is releasably coupled to the distal end of the adapter 120. In some aspects, the relief needle subassembly 140 and the adapter 120 may be connected to the syringe 110 in a single step. For example, the relief needle subassembly 140 and the adapter 120 may be initially connected or coupled with the proximal end of the needle 142 puncturing the multi-puncture septum 124 of the adapter 120. In other embodiments, the relief needle subassembly 140 and the adapter 120 may be coupled or connected to the syringe 110 in separate steps.

In the illustrated embodiment, the spacer 146 and the cap 144 comprise separate components which are connected, attached or otherwise coupled together using adhesives, mechanical attachment, heat welding or any other suitable means of coupling. In other embodiments, the cap 144 may be integrally formed with the spacer 146 by injection molding, for example. The spacer 146 includes two spacer arms 148a, 148b, which are configured to flex outward away from the adapter 120 to disengage the spacer arms 148a, 148b from the head portion of the connector 125 on the adapter 120. A groove 149 can be formed in each spacer arm 148a, 148b to receive the head portion of the connector 125. In some embodiments, an annular sleeve (not shown) may be wrapped around the interface between the relief needle subassembly 140 and the adapter 120 to prevent any fluids from leaking out from inside the cap 144.

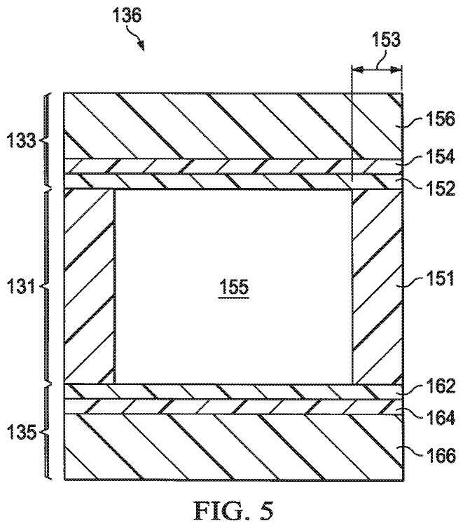

FIG. 5 is more detailed, cross-sectional view of the evacuated sampling chamber 136. The septa 133, 135 each include multiple layers bonded or otherwise attached to one another. The container 131 includes a hollow cylindrical or tubular body having a sidewall 151. The container 131 defines a chamber 155 configured to contain a volume of biological fluid. The sidewall 151 has a thickness 153. In some aspects, the thickness 153 may be sufficient to maintain a vacuum or negative pressure in the chamber 155 relative to the external environment.

In some aspects, the container 131 may be formed of a polymer material, glass, ceramic, metal or any other suitable material. For example, the container 131 may be formed of polyester, polyethylene, polycarbonate, polypropylene, nylon or any other suitable material or combination of materials. In some aspects, the container 131 includes a polymer body and a glass or ceramic coating on at least one of an interior surface or an exterior surface. In other embodiments, the container 131 includes a polymer body, such as polyester, where the thickness 153 is sufficient to prevent the sampling chamber 136 from losing the vacuum in the chamber 155 for a period of time. In some embodiments, the thickness 153 may be between 0.5 mm and 4 mm. In some embodiments, the sampling chamber 136 is configured to hold between 25 μL and 500 μL of fluid.

In the illustrated embodiment, the first septum 133 and the second septum 135 have a similar or identical construction. However, it will be understood that in other embodiments, the first septum 133 may have a different construction than the second septum 135. The first septum 133 includes an adherent layer 152, a metal foil layer 154, and an elastomeric layer 156. The adherent layer 152 may include a polymer material configured to bond and/or adhere to the material of the container 131.

In some embodiments, the adherent layer 152 includes an adhesive or mastic configured to provide a pressure activated and/or heat activated attachment with the container 131. In some embodiments, the adherent layer 152 includes a heat-activated material configured to bond, in response to applying heat, with a rim of the container 131 to provide a hermetic seal with the container 131. In some embodiments, the heat-activated material may include polyolefin, polyamide, polyester, polyurethane, and styrene butadiene copolymers. In some embodiments, the adherent layer 152 may have a thickness ranging from 0.005 mm to 0.1 mm.

The metallic foil layer 154 includes a metal foil bonded to the adherent layer 152. In some aspects, the metallic foil layer 154 may be adhered to the adherent layer 152 by an adhesive or other interfacial layer. The metallic foil layer 154 may include a metal having a thickness sufficient to maintain the vacuum or negative pressure within the chamber 155. In this regard, the metallic material of the foil layer 154 may be less permeable to gasses than some polymer materials. Accordingly, the metallic foil layer 154 improves the function of the seal to better maintain the pressure differential inside the chamber 155. The metallic foil layer 154 may include, for example, aluminum, gold, silver, copper, and/or alloys thereof. In some embodiments, the metallic foil layer 154 may have a thickness ranging from 0.01 mm to 0.2 mm.

The elastomeric layer 156 includes an elastomeric material having a thickness greater than the thicknesses of the adherent layer 152 and/or the metallic foil layer 154. In some embodiments, the thickness of the elastomeric layer 156 is greater than the sum of the thicknesses of the adherent layer 152 and the metallic foil layer 154. In this regard, the thickness of the elastomeric layer 156 may be greater than a length of a beveled opening of the needle 132. In this regard, the proximal end of the needle 132 may include a beveled or angled opening to facilitate puncturing of the septa 133, 135, and to prevent coring of the septa 133, 135.

If the thickness of the elastomeric layer 156 were significantly less than the length of the beveled opening of the needle 132, the beveled opening of the needle 132 may undesirably form a fluid path between the chamber 155 and the external environment, which may eliminate or reduce the negative pressure within the chamber 155 before the sampling chamber 136 is able to draw in a biological sample. Accordingly, the relatively large thickness of the elastomeric layer 156 maintains the hermetic seal while the needle is puncturing the septum 133. Further, in some aspects, elastomeric layer 156 may provide for a fluid seal with the exterior surface of the needle 132 so that the biological sample within the chamber 155 does not leak out into the external environment after the procedure. In some embodiments, the thickness of the elastomeric layer 156 may be between 0.2 mm and 3 mm, including values such as 0.4 mm, 0.5 mm, and 0.6 mm.

The second septum 135 also includes an adherent layer 162, and metallic foil layer 164, and an elastomeric layer 166. The layers 162, 164, 166 may be similar or identical to the layers 152, 154, 156 of the first septum 133. In other embodiments, one or more of the layers 162, 164, 166 may differ from the respective layers 152, 154, 156 and material, thickness, size or any other aspect.

In operation, once the relief needle subassembly 140 is connected to the adapter 120 and the syringe 110, the physician can first depress the plunger 112 using the push pad 114 and the finger grip 116. In this regard, the syringe 110, which may be a prefilled syringe, may include an air bubble or entrained air that the physician desires to expel. Further, the syringe 110 may contain a volume of the drug in excess of the desired dosage for the patient. Accordingly, the physician can puncture the multi-puncture septum 124 within the syringe 110 to expel the air bubble and the excess drug through the relief needle 142 and into the cap 144. In this regard, the cap 144 may form an enclosed space to contain the excess drug expelled through the relief needle 142. The relief needle 142 and cap 144 are then removed, and the syringe 110 and adapter 120 positioned within the working barrel 130 so that the multi-puncture septum 124 can be punctured a second time to deliver the drug to the patient's anatomy (e.g., vitreous humor).

With this in mind, the assembly 100 may be operated in a one-handed fashion, so that the physician may advance the drug syringe 110 and adapter 120 distally within the working barrel 130 using his or her hand 10, and support or stabilize the target anatomy (e.g., the eye 50) using the other hand. To this end, the physician may perform a combined biological sampling and injection procedure by advancing the syringe 110, adapter 120, and the evacuated sampling chamber 136 distally into the working barrel 130 toward a proximal end of the needle 132.

The physician may advance the syringe 110 distally by pressing on the plunger 112. The annular projection 137 is configured to engage at least a portion of the adapter 120 (e.g., o-ring 126) to alert the physician that the sampling chamber 136 is at a position at which the proximal end of the needle 132 has pierced the first septum 133 of the sampling chamber 136. The o-ring 126 and the proximal ring portion 121 may keep the adapter 120 centered within the working barrel 130 so that the proximal portion of the needle 132 impinges and punctures the multi-puncture septum 124 as the adapter 120 is moved distally within the working barrel 130.

At a first advanced position, the proximal end of the needle 132 has pierced the septum 133 of the sampling chamber 136, which draws in a volume of biological fluid into the chamber. The pressure differential between the inside of the sampling chamber 136 and the exterior environment may be sufficient to cause the biological sample (e.g., vitreous humor, blood, etc.) to automatically flow through the needle 132 and into the sampling chamber 136.

When the physician determines that the sampling chamber 136 is full, the physician continues to advance the syringe 110, adapter 120, and sampling chamber 136 distally into the working barrel 130 to a second advanced position until the distal end of the needle 132 exits the sampling chamber 136 by passing through the second septum 135. The needle 132 then pierces the multi-puncture septum 124 of the adapter 120 to place the distal end of the needle 132 in fluid communication with the drug inside the syringe 110. More specifically, fluid from inside the syringe 110 can now be expelled through the fluid channel 111 and into the proximal opening of the needle.

At the second advanced position, force applied to the plunger of the syringe 110 causes the plunger to force the drug out of the syringe 110 through the needle 132 and into the eye 50. Thus, the combined biological sampling and injection procedure is performed with one puncture of the patient's anatomy, and one multi-stage or stepwise motion in the same [distal] direction, increasing the efficiency and decreasing the complexity of the procedure.

As mentioned above, various components of the assembly 100 can be transparent—including the working barrel 130, the sampling chamber 136, and the syringe 110—to allow the physician to observe the progress of the biological sampling and injection procedure. This configuration, coupled with the lateral window(s) 128 in the adapter 120 and aforementioned tactile feedback features, provide for an easy viewable, repeatable process.

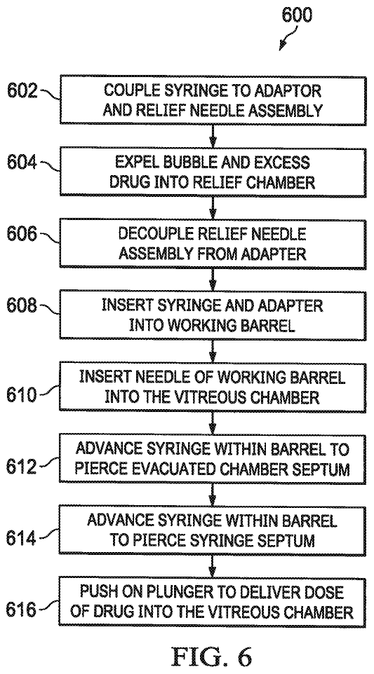

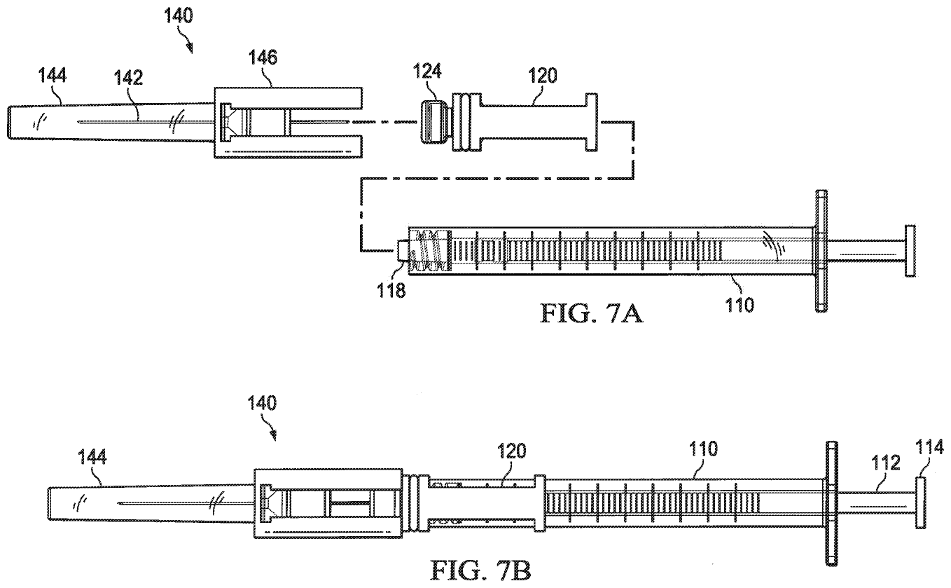

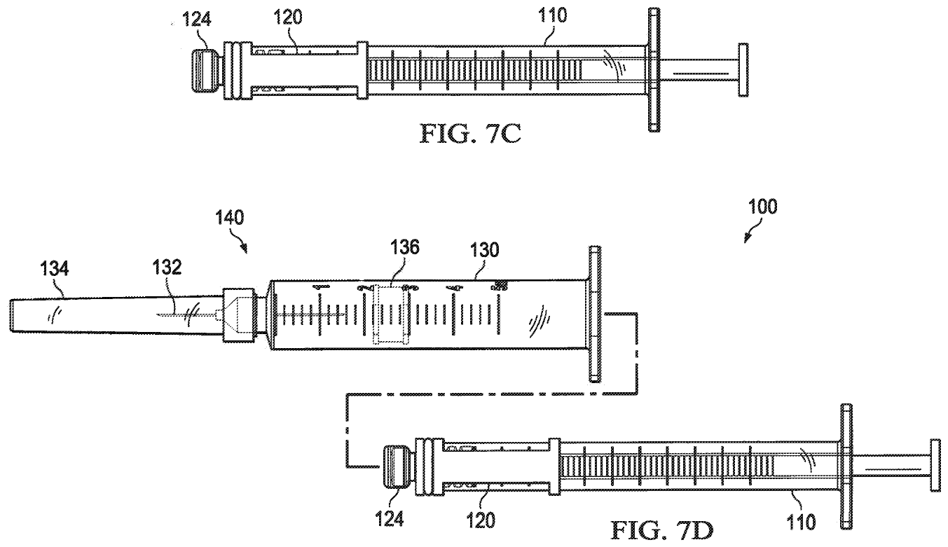

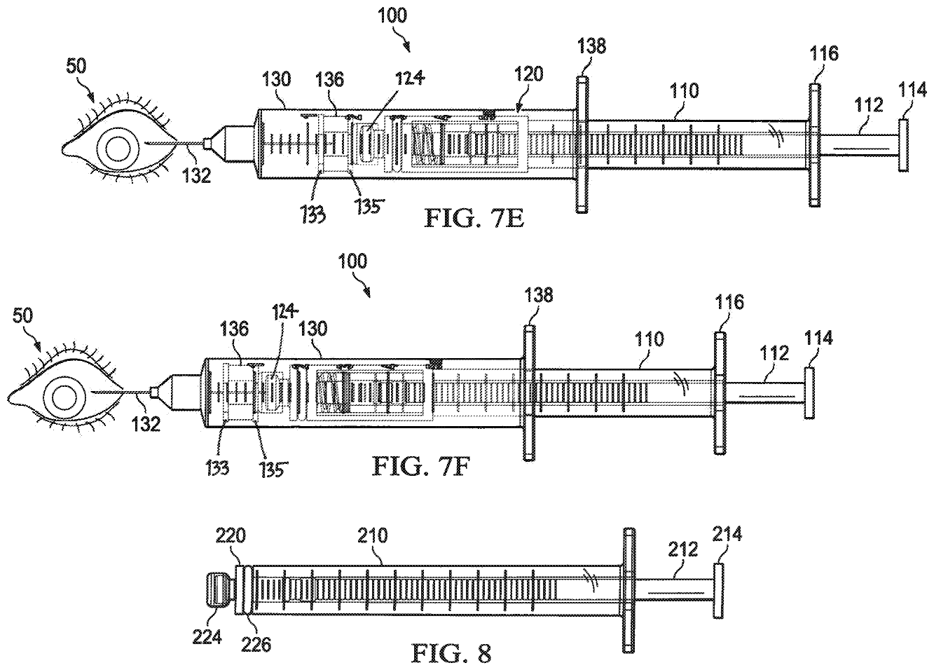

FIGS. 6 and 7A-7F illustrate a method 600 of performing a combined biological sampling and injection procedure. The method 600 may be performed using one or more of the devices, subassembliesor assemblies described above, including the combined biological sampling and injection subassemblies 100 illustrated in FIGS. 1 and 2.

Referring to FIGS. 6, 7A, and 7B, at step 602, the syringe 110 is coupled to the adapter 120 and relief needle assembly 140. As mentioned above, the coupling of the adapter 120 and the relief needle assembly 140 to the syringe 110 may be performed in a single step. In other embodiments, the adapter 120 may be first coupled to the distal end of the syringe 110, and the relief needle assembly 140 may be subsequently coupled to the adapter 120 and the syringe 110.

In either case, at step 604, the physician uses the plunger 112 of the syringe 110 to expel any bubbles, entrained air, and access drug from the syringe 110 through the relief needle 142 and into a drug collection chamber defined by the cap 144. In this way, the physician prepares the drug injection portion of the combined biological sampling and injection procedure.

Referring to FIGS. 6 and 7C, at step 606, the physician decouples the relief needle assembly 140 from the adapter 120. The adapter 120 remains coupled to the distal end of the syringe 110. The multi-puncture septum 124 of the adapter 120 automatically reseals the puncture hole created by the relief needle 142.

Referring to FIGS. 6 and 7D, at step 608, the physician inserts the syringe 110 and adapter 120 into a working barrel assembly, which includes the working barrel 130, the evacuated sampling chamber 136, the needle 132, and the protective cap 134. As noted, the adapter 120 may form a slidable interference fit with the working barrel 130. The resistance between the adapter 120 and the working barrel 130 may assist the physician to gradually depress the syringe 110, adapter 120, and sampling chamber 136 distally into the working barrel 130 toward the proximal end of the needle 132.

Referring to FIGS. 6 and 7E, at step 610, the physician inserts the needle 132 of the working barrel 130 into the patient's eye 50, and in particular into the vitreous chamber. At step 612, the physician advances the syringe 110 within and relative to the working barrel 130 so that the proximal end of the needle 132 pierces the first/distal septum 133 of the sampling chamber 136. To this end, the syringe 110 may be advanced distally into and relative to the working barrel 130 by pressing on the push pad 114 of the plunger 112. Because the multi-puncture septum 124 of the adapter 120 is no longer pierced, pushing on the plunger 112 causes the entire syringe 110 to advance distally into the working barrel 130, rather than causing additional drug to be expelled from the syringe 110. The physician may use the push pad 114 and the finger grip 138 of the working barrel 130 to advance the syringe 110, adapter 120, and sampling chamber 136 distally in the working barrel 130.

The proximal end of the needle 132 may include a beveled or angled opening as described above. In this regard, a length of the beveled opening may be less than a thickness of the first septum 133 of the sampling chamber 136. When the proximal end of the needle 132 enters the interior of the evacuated sampling chamber 136, the pressure differential causes vitreous humor to automatically flow through the needle 132 and into the sampling chamber 136. As noted, the working barrel 130 and the sampling chamber 136 are formed of transparent or translucent materials so that the physician can observe the vitreous humor flowing into the sampling chamber 136.

Furthermore, the annular projection 137 (see FIG. 2) on the working barrel 130 provides the physician with tactile feedback when the proximal end of the needle 132 has punctured the sampling chamber 136. The projection 137 may be configured to, for example, interact with the o-ring 126 of the adapter 120 to provide haptic feedback. It will be appreciated that instead of an annular projection the tactile features can include a detent, ridge, grooveor any other structural features suitable for providing tactile feedback to the physician regarding the position of the syringe 110, adapter 120, and/or sampling chamber 136 relative to the working barrel 130 and proximal end of the needle 132.

Referring to FIGS. 6 and 7F, at step 614, once the desired volume of vitreous humor is collected the physician advances the syringe 110, adapter 120, and sampling chamber 136 within the working barrel 130 until the proximal end of the needle 132 pierces the multi-puncture septum 124 of the syringe 110. In some aspects, step 614 may include the physician pushing on the push pad 114 of the plunger 112 until the sampling chamber 136 is stopped by the geometry of the distal end of the working barrel 130. As the sampling chamber 136 advances distally into the working barrel 130, the proximal end of the needle 132 punctures the second/proximal septum 135 of the sampling chamber 136 and then punctures the multi-puncture septum 124 of the adapter 120.

As a result, the proximal end or opening of the needle 132 is placed in fluid communication with the drug inside the syringe 110. Accordingly, as the physician continues to push on the push pad 114, the force of the plunger 112 now causes the drug within the syringe 110 to be expelled through the needle 132 and into the vitreous humor of the patient's eye 50. The volume of drug injected into the patient may be equal toor approximately equal to the volume of the biological sample material drawn into the sampling chamber 136.

FIG. 8 illustrates another example, prefilled syringe 210 having a distal connector 220, which includes a multi-puncture septum 224 and an o-ring 226. In this regard, instead of using an adapter, such as the adapter 120 shown in FIGS. 2-7F above, the multi-puncture septum 224 is incorporated into the syringe 210 such that no adapter is necessary to perform the combined biological sampling and injection procedures described above. The multi-puncture septum 224 may be crimped onto a head of the distal connector 220. The syringe 210 can be operated with the working barrel 130 and relief needle subassembly 140 in the same manner as described above with respect to the syringe 110.

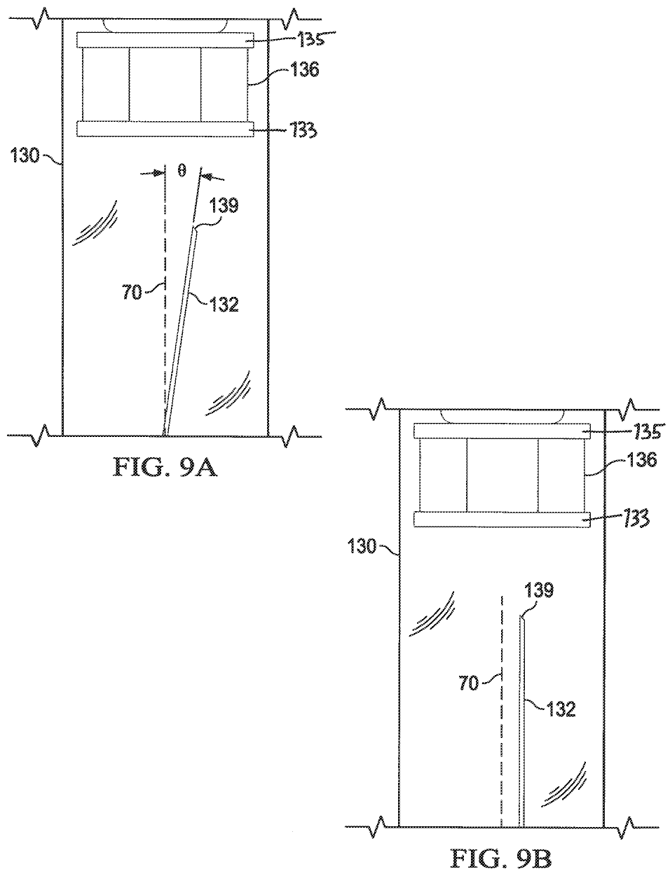

FIGS. 9A and 9B illustrate a particular configuration for the needle 132 of the working barrel 130. As noted, the needle 132 may include an angled or beveled opening 139 at the proximal end. The angled or beveled opening 139 may facilitate the puncturing of the needle 132 into the sampling chamber 136. For example, the beveled opening 139 may provide a sharp point for the needle 132 to puncture the septa 133, 135 of the sampling chamber 136 while reducing or eliminating coring of the septa 133, 135.

However, the angled surface of the beveled opening 139 may cause the needle 132 to deflect away from its longitudinal axis 70. Accordingly, in the embodiment shown in FIG. 9A, the needle 132 is initially deflected or pre-deflected to compensate for the deflection caused by the beveled opening 139 penetrating the septa 133, 135 of the sampling chamber 136. In particular, the needle 132 can be angularly deflected or tilted away from the longitudinal axis 70 by an angle θ.

In another example shown in FIG. 9B, the needle 132 can be laterally deflected or shifted away from the longitudinal axis 70. In either case, the angular or lateral deflection of the needle 132 may be determined or configured such that the beveled opening 139 puncturing the first septum 133 of the evacuated chamber 136 causes an angular and/or lateral deflection of the proximal portion of the needle 132 toward the longitudinal axis 70.

It will be understood that the devices, assemblies, and subassemblies described above may be modified from the specific embodiments illustrated without departing from the scope of the present disclosure. For example, although the adapter 120 is described as having a multi-puncture septum 124, other types of connectors and/or interfaces may be used other than or in addition to a multi-puncture septum. For example, in some embodiments, the adapter 120 may include a single puncture septum. In other embodiments, the adapter 120 may include a valve configured to receive a needle or cannula to create a fluid path between the syringe 110 and the needle 132. Further aspects and/or details of the assemblies and devices described above can be found in U.S. patent application Ser. No. 17/319,742, filed May 13, 2021, the entirety of which is incorporated by reference herein.

Persons skilled in the art will recognize that the devices, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure.

Accordingly, it is appropriate that the appended claims be construed broadly and in a manner consistent with the present disclosure.

What is claimed is:

1. A sampling device, comprising:
a container comprising a body defining a chamber and an opening to the chamber; and
a septum bonded to the container to cover the opening and hermetically seal the chamber, the septum comprising:
an adherent layer having a first thickness and comprising a material configured to adhere to a rim of the container to provide a hermetic seal with the container;
a metallic foil layer having a second thickness and being coupled to the adherent layer; and
an elastomeric layer having a third thickness and being coupled to the metallic foil layer to position the metallic foil layer between the adherent layer and the elastomeric layer, wherein the third thickness is greater than the sum of the first thickness and the second thickness.

2. The sampling device of claim 1,
wherein the opening of the container is a distal opening,
wherein the container further includes a proximal opening opposite and aligned with the distal opening, and
wherein the sampling device further comprises a proximal septum bonded to the container to cover the proximal opening, the proximal septum comprising:
an adherent layer comprising a material configured to adhere to a proximal rim of the container to provide a hermetic seal with the container;
a metallic foil layer coupled to the adherent layer of the proximal septum; and
an elastomeric layer coupled to the metallic foil layer of the proximal septum to position the metallic foil layer of the proximal septum between the adherent layer and the elastomeric layer of the proximal septum.

3. The sampling device of claim 2, wherein the adherent layer of the proximal septum has a fourth thickness, the metallic foil layer of the proximal septum has a fifth thickness, and the elastomeric layer of the proximal septum has a sixth thickness, wherein the sixth thickness is greater than the sum of the fourth thickness and the fifth thickness.

4. The sampling device of claim 1, wherein the adherent layer comprises a material configured to bond with the rim of the container in response to applying heat.

5. The sampling device of claim 1, wherein the adherent layer comprises an adhesive.

6. A system for use with a syringe for obtaining a biological sample and delivering a pharmaceutical agent to a patient, the system comprising:
a barrel comprising a hollow body defining an interior region;
an adapter positioned within the interior region and having a proximal connecter configured to couple the adapter to a distal end of the syringe, a first septum being disposed at a distal end of the adapter; and
an evacuated container slidably received within the interior region distally from the adapter and having a body defining a chamber configured to contain the biological sample and an opening extending to the chamber, wherein a second septum covers the opening; and
a two-sided needle having a proximal end disposed within the barrel distally from the evacuated container and a distal end positioned for insertion into the patient, wherein the evacuated chamber is movable within the barrel to a first position placing the proximal end of the needle within the chamber to draw the biological sample from the patient into the chamber and to a second position in which the proximal end of the needle passes entirely through the evacuated chamber and through the first septum to deliver the pharmaceutical agent from the syringe to the patient.

7. The system of claim 6, wherein the evacuated container is movable in the same direction relative to the barrel to reach both the first and second positions.

8. The system of claim 6, wherein the opening of the container is a distal opening and the second septum comprises:
an adherent layer comprising a material configured to adhere to a distal rim of the container to provide a hermetic seal with the container;
a metallic foil layer coupled to the adherent layer of the second septum; and
an elastomeric layer coupled to the metallic foil layer of the second septum to position the metallic foil layer of the second septum between the adherent layer and the elastomeric layer of the second septum.

9. The system of claim 8, wherein the adherent layer has a first thickness, the metallic foil layer has a second thickness, and the elastomeric layer has a third thickness, wherein the third thickness is greater than the sum of the first thickness and the second thickness.

10. The system of claim 8, wherein the evacuated container further comprises a third septum bonded to the container at a proximal opening opposite and aligned with the distal opening to cover the proximal opening, the third septum comprising:
an adherent layer comprising a material configured to adhere to a proximal rim of the container to provide a hermetic seal with the container;
a metallic foil layer coupled to the adherent layer of the third septum; and
an elastomeric layer coupled to the metallic foil layer of the third septum, wherein the metallic foil layer of the third septum is disposed between the adherent layer and the elastomeric layer of the third septum.

11. The system of claim 10, wherein the adherent layer of the third septum has a fourth thickness, the metallic foil layer of the third septum has a fifth thickness, and the elastomeric layer of the third septum has a sixth thickness, wherein the sixth thickness is greater than the sum of the fourth thickness and the fifth thickness.

12. The system of claim 6, wherein the adapter comprises an o-ring around a perimeter of the adapter, wherein an outer diameter of the o-ring corresponds to an inner diameter of the barrel.

13. The system of claim 12, wherein the barrel further comprises a tactile feature within the interior region and cooperating with the o-ring to indicate that the evacuated container is at the first position.

14. The system of claim 6, wherein the proximal end of the two-sided needle comprises a beveled opening.

15. The system of claim 14,
wherein the barrel defines a longitudinal axis,
wherein at least the proximal end of the two-sided needle is offset from the longitudinal axis, and
wherein the beveled opening is configured to cause, in response to puncturing the second septum of the evacuated container, a deflection of the proximal portion of the two-sided needle toward the longitudinal axis.

16. An adapter configured to couple to a syringe, the adapter comprising:
a receptacle configured to receive a portion of the syringe;
a proximal connector on the receptacle configured to mate with a corresponding connector feature of the syringe, wherein the proximal connector is a first type of connector, and wherein the proximal connector includes a channel extending along a needle axis; and
a septum disposed at a distal end of the receptacle configured to mate with a needle assembly that includes a second type of connector different from the first type of connector.

17. The adapter of claim 16, wherein the receptacle comprises a cylindrical body having at least one lateral window extending therethrough.

18. The adapter of claim 16, wherein the proximal connector comprises a female luer-lock connector and wherein the septum comprises a multi-puncture septum.

19. The adapter of claim 16, wherein the receptacle has a first width and the adapter further comprises a distal neck coupled to the septum and having a second width smaller than the first width.

20. The adapter of claim 16, further comprising a relief needle assembly comprising:
a needle comprising a proximal portion and a distal portion;
a cap positioned around the distal portion; and
a spacer coupled to the cap and extending proximally from the cap, wherein the spacer is disposed at least partially around the proximal portion of the needle and configured to releasably couple to the distal connector.

21. The adapter of claim 20, wherein the spacer is configured to releasably couple to the distal connector such that the proximal portion of the needle punctures the septum.
